(19) United States
(12) Patent Application Publication
(10) Pub. No.: US 2004/0158186 A1
(43) Pub. Date: Aug. 12, 2004

(54) Indicia-bearing adhesive bandage and method of vaccination employing same

(76) Inventor: Dana Hall, Glen Falls, NY (US)

Correspondence Address: Dana Hall, 19 Cunningham Avenue, Glens Falls, NY, 12801-2606, US

(21) Appl. No.: 10/365,215

(22) Filed: Feb. 12, 2003

(51) Int. Cl. A61F 13/00

(52) U.S. Cl. 602/48

(57) ABSTRACT

An adhesive bandage applied to a vaccination site for the purpose of maintaining the cleanliness of the site possesses indicia on its exposed, or viewable, surface identifying the specific vaccine which was administered at the site thus immediately and unambiguously identifying the vaccine in the event identification of the vaccine should subsequently become necessary or desirable, e.g., in the event of an adverse reaction to the vaccine.

### BACKGROUND OF THE INVENTION

(1) Field of the Invention

This invention relates to the fields of surgical adhesive bandages and vaccination methods utilizing such bandages for the protection of a vaccine injection site.

(2) Description of Related Art

In a typical vaccination procedure, a measured quantity of vaccine is administered to an intended recipient by intramuscular or subcutaneous injection via hypodermic syringe at a preselected site on the recipient's skin which has been freshly prepared by scrubbing with alcohol or an iodophor such as povidone to minimize the risk of microbial infection and, following administration of the vaccine, an adhesive surgical bandage is applied to the injection site to maintain the cleanliness of the site for a suitable period of time, e.g., up to about 24 hours or so.

Several vaccinations may be separately but concurrently administered to a single individual at multiple injection sites, such frequently being the case with infants and young children. Occasionally, a local reaction, e.g., an abscess, may soon develop at an injection site. When this occurs, it may be difficult to determine which of the concurrently administered vaccines caused the adverse reaction. Even where only one vaccine has been administered, it may be difficult for a physician or other medical practitioner to identify the particular vaccine which caused the adverse reaction without resorting to a possibly inconvenient and time-consuming investigation into the circumstances of the administration of the vaccine. While most adverse reactions following vaccination require no treatment, some individuals may need evaluation, therapy and/or exemption from further vaccine doses. Ideally, there should be no potential for misidentifying a vaccine which has caused an adverse reaction in a patient, or a delay of any sort in identifying the vaccine which has caused the reaction.

### BRIEF SUMMARY OF THE INVENTION

In accordance with the present invention, there is provided an adhesive bandage for application to the skin of a vaccination recipient at the site of vaccination, the bandage having a skin adherent surface and an exposed surface when the bandage is in adherent contact with the vaccination recipient's skin, the exposed surface bearing indicia identifying the vaccine which was administered by injection at the site.

Further in accordance with this invention, there is provided a method of vaccination and a kit for carrying out a vaccination procedure which utilizes the aforedescribed indicia-bearing adhesive bandage.

Use of the indicia-bearing adhesive bandage of this invention in connection with the administration of a vaccine results in a number of benefits and advantages over present vaccine administration procedures in which an unlabeled or otherwise unidentified adhesive bandage is applied to a vaccine injection site.

When several different vaccines are to be administered concurrently to a single recipient, the indicia-bearing adhesive bandage serves as a cross-reference for the correct vaccine to be administered when placed with the hypodermic syringe and vial of vaccine. The use of the indicia-bearing adhesive bandage also functions as notification to both the vaccine recipient (or his/her guardian or caregiver) and medical personnel that a particular vaccine has been administered thus reducing the risk of an accidental re-administration of the vaccine or failure to administer the vaccine at all. And in the case of a vaccine that has resulted in a more or less severe adverse reaction in the recipient, the use of the indicia-bearing adhesive bandage herein identifying the administered vaccine enables the vaccine recipient (or his/her guardian or caregiver) and medical practitioner to immediately identify the vaccine which caused the reaction, a benefit that is particularly desirable where several vaccines have been concurrently administered to an individual.

### BRIEF DESCRIPTION OF THE DRAWINGS

FIG. 1 illustrates an adhesive bandage in accordance with the invention bearing the identification of a particular vaccine printed on its exposed, i.e., nonadhesive, side;

FIG. 2 illustrates another embodiment of an adhesive bandage in accordance with the invention bearing an adhesive label applied to its exposed surface with the identification of a particular vaccine printed on the viewable surface of the label;

FIG. 3 illustrates a kit for use in a vaccination procedure which includes a vial of vaccine, an adhesive bandage in accordance with FIG. 1 or FIG. 2 and, as optional components, a hypodermic syringe and injection site preparative.

### DETAILED DESCRIPTION OF THE INVENTION

The adhesive bandage to which the identification of a particular vaccine is applied to provide the indicia-bearing adhesive bandage of this invention can be any one of several known and conventional types. In general, the adhesive bandage will possess a flexible backing, or strip, one side of which possesses a layer of adhesive and a gauze pad (the skin adherent side of the bandage) and the other side of which is exposed to view.

The vaccine-designating indicia can be applied to the exposed, or viewable, surface of the adhesive bandage in a variety of ways, e.g., it can be handwritten or printed thereon or it can be applied thereto as a separate adhesive label. The indicia can be made up of text, graphic symbols or other pictorial elements, color or a combination of any of the foregoing. Text is generally preferred for its ability to immediately and unambiguously convey the desired information regarding the identity of the vaccine and, optionally, other particulars as well. Thus, in addition to the identification of the vaccine to be administered, the indicia can convey other useful information such as the date of the vaccine, the brandname/tradename of the vaccine and/or its manufacturer, the quantity of vaccine administered, etc. The vaccine can be identified by its generic name and/or by its brandname/tradenames. When adhesive labels are employed, the labels can be preprinted or they can be printed when and as needed on adhesive label stock in a computerized printing operation.

In accordance with the method of vaccination of the present invention, following administration of the vaccine, the site of injection is covered by the indicia-bearing adhesive bandage herein, or if the identity of the vaccine that has been administered has not yet been applied to the exposed surface of the bandage, such is then carried out, e.g., by a handwritten entry or entries or by application of an adhesive label bearing the pertinent information.

The following table lists some of the vaccines that can be administered to an intended vaccine recipient in accordance with the invention.

| Vaccine | Brandname/Tradename | Manufacturer |
|---|---|---|
| DtaP | Tripedia ® | Aventis Pasteur |
| DtaP | Infanrix ® | Glaxo SmithKline |
| DtaP - Hib | TRiHIBit ® | Aventis Pasteur |
| e-IPV | IPOL | Aventis Pasteur |
| Hepatitis B - Hib | COMVAX ® | Merck |
| Hepatitis A Pediatric | VAQTA ® | Merck |
| Hepatitis A Pediatric | Havrix ® | Glaxo SmithKline |
| Hepatitis A Adult | VAQTA ® | Merck |
| Hepatitis A Adult | Havrix ® | Glaxo SmithKline |
| Hepatitis A - Hepatitis B | Twinris ® | Glaxo SmithKline |
| Adult | | |
| Hepatitis B | ENGERIX B ® | Glaxo SmithKline |
| Preservative Free | | |
| Pediatric/Adolescent | | |
| Hepatitis B | RECOMBIVAX HB ® | Merck |
| Preservative Free | | |
| Pediatric/Adolescent | | |
| Hepatitis B 2 dose | RECOMBIVAX HB ® | Merck |
| Adolescent (11-15) | | |
| Hepatitis A-Hepatitis B | Twinris ® | Glaxo SmithKline |
| 18 year old | | |
| Hepatitis B - Adult | RECOMBIVAX HB ® | Merck |
| Hepatitis B - Adult | ENGERIX B ® | Glaxo SmithKline |
| Hib | PedvaxHIB ® | Merck |
| Hib | HibTITER ® | Wyeth/Lederle |
| Hib | ActHIB ® | Aventis Pasteur |
| Influenza | Fluzone ® | Aventis Pasteur |
| Influenza | Fluvirin ® | GIV (Evans) |
| MMR | MMR ® II | Merck |
| Measles | Attenuvax ® | Merck |
| Mumps | Mumpsvax ® | Merck |
| Pediarix | Pediarix ™ | Glaxo SmithKline |
| Pneumococcal | Prevnar ® | Wyeth/Lederle |
| 7-valent (pediatric) | | |
| Pneumococcal | Pneumovax 23 ® | Merck |
| 23-valent (adult) | | |
| Rubella | Meruvax II ® | Merck |
| Varicella | Varivax ® | Merck |

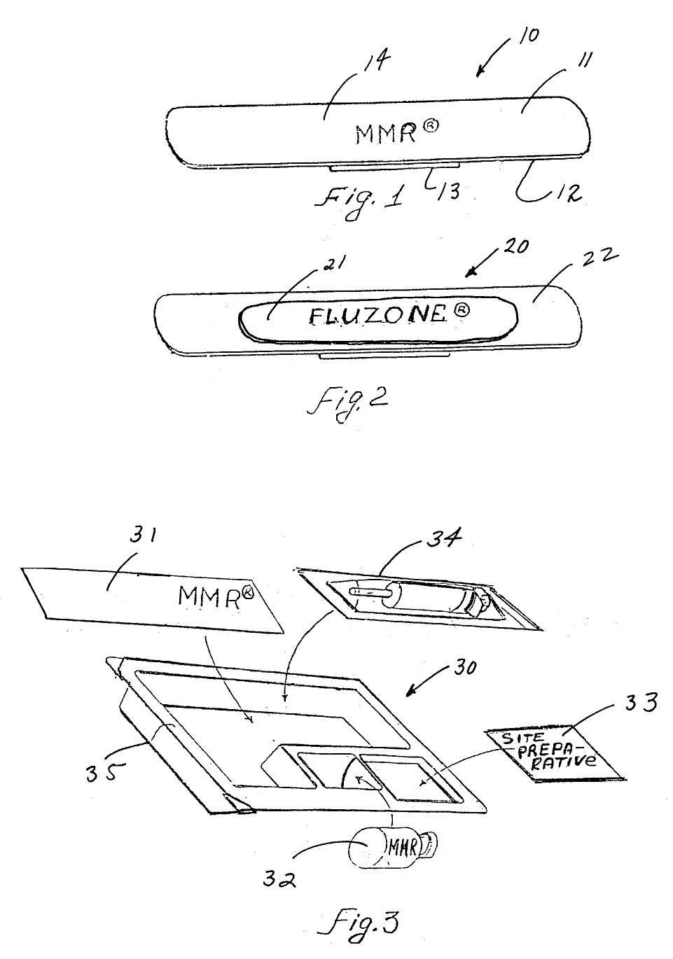

Embodiments of an indicia-bearing adhesive bandage in accordance with the present invention are described in FIGS. 1 and 2. As illustrated in FIG. 1, adhesive bandage 10 includes a front, or viewable, side 11, and an adhesive-coated back side 12 by which the bandage is applied, and made to adhere, to a site of vaccine injection on the recipient's skin. Adhesive bandage 10 includes a gauze pad 13 which, in the usual application of the bandage, is approximately centered upon the vaccine injection site. Central region 14 of the front side 11 of bandage 10 bears pre-printed indicia identifying the vaccine which is to be administered to the vaccination recipient, in this case, MMR® (measles, mumps and rubella combination vaccine).

Adhesive bandage 20 of FIG. 2 illustrates another embodiment of the indicia-bearing adhesive bandage herein with the identification of the vaccine (FLUZONE®, influenza vaccine) being placed on an adhesive label 21 applied to the front side 22 of the bandage.

Kit 30 shown in FIG. 3 contains everything that is needed to carry out a single vaccine injection including an aseptically packaged indicia-bearing adhesive bandage 31, e.g., the embodiment shown in FIG. 1 or FIG. 2, a vial of vaccine 32, an aseptically packaged site preparative 33, e.g., a foil-wrapped applicator containing povidone, and aseptically packaged hypodermic syringe 34, all nestled within suitably configured compartments of plastic tray 35 which is eventually sealed with a peel-away cover (not shown).

What is claimed is:

1. An adhesive bandage for application to the skin of a vaccination recipient at the site of vaccination, the bandage having a skin adherent surface and an exposed surface when the bandage is in adherent contact with the vaccination recipient's skin, the exposed surface bearing indicia identifying the vaccine which was administered by injection at the site.

2. The adhesive bandage of claim 1 wherein the indicia identify the vaccine as a member of the group consisting of measles, mumps and rubella vaccine, diphtheria, tetanus and acellular pertussive vaccine, hepatitis B vaccine, inactive polio vaccine, chicken pox vaccine, pneumococcal conjugate V-7 vaccine and meningitis virus vaccine.

3. The adhesive bandage of claim 1 wherein the indicia is text, graphics, color or any combination thereof.

4. The adhesive bandage of claim 2 wherein the indicia is text, graphics, color or any combination thereof.

5. The adhesive bandage of claim 3 wherein the text, graphics or color is present directly on the exposed surface of the bandage.

6. The adhesive bandage of claim 5 wherein the text, graphics or color is printed.

7. The adhesive bandage of claim 5 wherein the text, graphics or color is applied by hand.

8. The adhesive bandage of claim 3 wherein the text, graphics or color is present on a label adhesively applied to the exposed surface of the bandage.

9. The adhesive bandage of claim 8 wherein the text, graphics or color is printed.

10. The adhesive bandage of claim 8 wherein the text, graphics or color is applied by-hand.

11. A method of vaccination which comprises:
a) administering a vaccine by injection to an intended recipient thereof at a preselected vaccination site; and,
b) applying an adhesive bandage to the vaccination site, the adhesive bandage having a skin adherent surface and an exposed surface when the bandage is in adherent contact with the vaccination recipient's skin, the exposed surface bearing indicia identifying the vaccine which was administered by injection at the site, the indicia being applied to the adhesive bandage prior to or following application of the adhesive bandage to the site of injection.

12. The method of claim 11 wherein the indicia identify the vaccine as a member of the group consisting of measles, mumps and rubella vaccine, diphtheria, tetanus and acellular pertussive vaccine, hepatitis B vaccine, inactive polio vaccine, chicken pox vaccine, pneumococcal conjugate V-7 vaccine and meningitis virus vaccine.

13. The method of claim 11 in which at least two different vaccines are separately and concurrently administered to the intended recipient with separate adhesive bandages being applied to each vaccination site.

14. A kit for use in a vaccination procedure, the kit comprising:
a) at least one sealed container of vaccine; and,
b) at least one adhesive bandage for application to the skin of a vaccination recipient at the site of vaccination, the bandage having a skin adherent surface and an exposed surface when the bandage is in adherent contact with the vaccination recipient's skin, the exposed surface bearing indicia identifying the vaccine to be administered by injection at the site.

15. The kit of claim 14 wherein the indicia identify the vaccine as a member of the group consisting of measles, mumps and rubella vaccine, diphtheria, tetanus and acellular pertussive vaccine, hepatitis B vaccine, inactive polio vaccine, chicken pox vaccine, pneumococcal conjugate V-7 vaccine and meningitis virus vaccine.

16. The kit of claim 15 further comprising a hypodermic syringe for injecting the vaccine.

17. The kit of claim 16 further comprising an injection site preparative.
